(19) United States
(12) Patent Application Publication
(10) Pub. No.: US 2008/0300563 A1
(43) Pub. Date: Dec. 4, 2008

(54) Absorbent Article with Improved Elastic Means

(75) Inventors: Carolyn Berland, Molndal (SE); Maria Fernkvist, Molndal (SE); Ingrid Gustafson, Asa (SE)

Correspondence Address: DRINKER BIDDLE & REATH (DC), 1500 K STREET, N.W., SUITE 1100, WASHINGTON, DC, 20005-1209, US

(73) Assignee: SCA Hygiene Products AB, Goteborg (SE)

(21) Appl. No.: 12/094,394

(22) PCT Filed: Dec. 23, 2005
(86) PCT No.: PCT/SE05/02046

§ 371 Date: May 20, 2008

(51) Int. Cl. A61F 13/15

(52) U.S. Cl. 604/367; 604/385.06; 604/385.27; 604/392

(57) ABSTRACT

The invention concerns an absorbent article such as a diaper, an incontinence product, a sanitary napkin, a panty liner or the like, wherein one or several parts (103,203: 104,204: 105,205) of said article comprise elastic means (116,216: 117,217; 119,219) which hold a skincare agent in their structure. The skincare agent is arranged to be released when the elastic means (116,216: 117,217; 119,219) is stretched during use of the absorbent article and the elastic means (116,216: 117,217; 119,219) absorbs less than 20% water or urine

### TECHNICAL FIELD

The invention refers to an absorbent article such as a diaper, an incontinence product, a sanitary napkin, a panty liner or the like, wherein one or several parts of said article comprise elastic means which hold a skincare agent in their structure.

### BACKGROUND ART

The main purpose of absorbent articles, such as diapers, incontinence protectors and sanitary products, is to absorb different body secretions, such as urine, faeces, menstrual fluid and the like. In order to ensure that the articles lie sealingly against the user, the absorbent articles are usually provided with various types of elastic, such as waist elastic or leg elastic. It is also common to arrange various elastic arrangements in connection with the crotch area in order to prevent side leakage from the region which will bear the brunt of the body secretions. The elastic thus contributes towards ensuring that the absorbent article lies so closely against the user's body that neither body secretions nor odour leak out to the environment.

As the aim is that the article is closely tightened during use, problems with chafing from the parts of the absorbent article that lie most tightly against the user's body can easily arise. Further problems arise when body secretions are emitted, which body secretions can irritate adjacent skin surfaces. In addition, chafing leads to the skin being more susceptible and more sensitive to negative influences from excrement and urine and those environments which follow as a result in a product when it is subjected to this.

These problems can be solved by manually applying skin cream to affected areas when changing the product. However, a disadvantage of this procedure is that the product is usually not changed until body secretions have been emitted, which means that the article will chafe for long periods between changes. In addition, it is also easy to forget to apply skin cream or to apply it hastily so that creams are spread carelessly over the skin surfaces.

Furthermore, in those cases where ointment or lotion are applied to the skin before the absorbent article is attached to the user, some of the substance can go over and penetrate into the surface layer of the absorbent article and prevent the absorbent body from taking up urine, for example. The manual application also means that when the applied skincare agent has been completely absorbed by the skin, it has been used up. In addition to this, a carer may feel that applying a skincare agent directly to the skin is uncomfortable and unhygienic, not only with regard to the fact that it can be sticky to use ointment or the like, but also because the user may, for example, have infected sores and the user may have scabs that the carer must touch even though he or she does not want to. Similarly, the user may also not want a carer to touch the wound or the sensitive skin directly with his or her hands.

In order to solve these problems, several attempts have been made to provide absorbent articles with various types of creams, ointments, lotions, or the like. One example is WO 96/16682 in which document the above-mentioned problem is solved by adding therapeutic or protective components by means of lotion which has been laid on a liquid-permeable surface layer. It is even claimed that the diaper according to WO 96/16682 solves accompanying problems such as impaired admission into the liquid-permeable surface layer by using hydrophilic lotion on the surface layer.

However, the diaper described in WO 96/16682 is connected with several problems. During each process where lotion is applied at high speed onto a liquid-permeable material, it is in principle impossible to avoid partially blocking at least some of the pores on the liquid-permeable surface layer. Attempts to avoid or in any case minimise this problem can be made by applying the lotion in lines or specific patterns on the liquid-permeable surface layer. However, the problem of blocked pores remains in the application pattern.

Further problems are that lotion can migrate down from the surface layer, or from other parts of the absorbent article that have had ointment or lotion applied to them, and down through the pores thus preventing the absorbent body from taking up liquid. This problem is remedied by manufacturing lotions and lotion stabilisers with specific melt temperatures, which should preferably be completely or at least partially solid at room temperature and should begin to melt preferably just under body temperature.

Despite modification of the lotion, the product is subjected before use, for example during transport and storage, to conditions such as more extreme temperatures, which means that the lotion can nevertheless migrate at least partially and thus impair admission into the absorbent article by blocking the pores of the surface layer and thus both indirectly also the absorbent core. Migration of lotion to the absorbent core can also lead to a direct impairment of the absorption capacity. Migration problems arise, for example, when the product lies in a warm warehouse or is stored under other warm conditions which occur in everyday life. Accordingly, there are situations which it is not possible to protect oneself from merely by modifying the lotion so that it obtains a specific melt temperature.

WO 99/22684 describes a web with at least two different sorts of skincare material. The web is attached to various places on a diaper, e.g. the surface layer, the liquid barriers or the like. No particular attention is given to the problem of blockage of pores as a result of migration in this case either.

EP 1 358 863 describes lotion-containing hydrogels with a certain elasticity and their use in absorbent articles. The lotion is built in to the chemical structure of the hydrogels and is emitted continuously by means of migration in the material. In an absorbent article this means that there is a risk of the lotion migrating from the hydrogel to other parts of the article during transport and storage of the article. This means of course that the active component does not obtain the intended effect when the article is used. Furthermore, wrongly placed lotion can have a negative effect on the fluid-receiving capacity and the absorption capacity of the absorbent article by changing the wettability on the liquid-permeable surface layer and the absorbent material of the article. Hydrogels are also highly water-absorbent, which means that during use in an absorbent article they take up emitted body fluid and thus become wet so that they make a user's skin wet or damp when they lie against it. This is unsuitable as damp skin is more susceptible to chafing and other irritation.

Consequently, a purpose of the invention is to offer a solution involving transferring of skincare agent to particularly vulnerable areas of skin. The invention is further intended to solve the problem of packaging and storage in such a way that the skincare agent does not affect the absorbent article. The invention provides the possibility to obtain sufficient transfer of, for example, lotion without the lotion adversely affecting the admission into the absorbent article, or the absorption capacity of the absorbent article. There exists a need for a solution which enables the use of skincare agent in an absorbent article, or the like, both for preventative purposes and to soothe rashes or skin irritations or facilitate the care of pressure sores, bedsores, or the like. Furthermore, there is a need for a means which offers more variations for skincare with predetermined placing than with lotion-coated surface areas in diapers. In addition, there exists a need for an absorbent article which minimises chafing between the sensitive skin (damaged skin or where prevention of damage to the skin is desired) and the absorbent article, one way of minimising chafing being to use means that can reduce the friction between the product and the skin. Thus, there exists a need for means which not only transfers skincare agent but also minimises chafing in the affected areas. In addition to this, there is a need for a cost-effective solution to the above-mentioned problems.

### DESCRIPTION OF THE INVENTION

An absorbent article of the type mentioned in the introduction, which essentially eliminates the problems that exist with previously known such absorbent articles, has been achieved with the present invention.

An article produced in accordance with the invention is chiefly characterised in that the skincare agent is arranged to be released when the elastic means is stretched during use of the absorbent article and in that the elastic means absorbs less than 20% water or urine.

Arranging skincare agent in the structure of the functional elastic means comprised in the absorbent article, which elastic means are characterised by, among other things, their ability to be stretched, facilitates the discharge of skincare agent out of the elastic means only when the absorbent article is used. In addition, the elastic means can be treated so that the skincare agent is not released until stretching or extension of the elastic means exceeds a certain threshold value. The conventional elastic means in the absorbent article have been treated during the production process, in unstretched and unextended condition, in order to take up skincare agent in their pore structure by pressing or swelling with the skincare agent.

The elastic means which have been filled with skincare agent can be fixed to the absorbent article in this non-prestretched condition. However, it is common for the elastic means to be stretched out during fixing. In order to avoid unintentional release during manufacture of the absorbent article, it is therefore expedient that the release does not occur before a certain threshold value for extension is reached. Another way to ensure that the skincare agent stays in the elastic means even when stretched during manufacture of the absorbent article is to use skincare agents that are in solid form or are viscous at room temperature but melt and become fluid or less viscous when warmed to body temperature. Such skincare materials are activated and can be released only when the absorbent article is used and is subjected to simultaneous warming and extension. Due to the fact that the skincare agent is held in place mechanically in the filled elastic means, it is not affected by only warming during transport and storage or only stretching when the absorbent article is manufactured. Therefore, the elastic means filled with skincare agent keeps the skincare agent safely stored until use of an absorbent article comprising the elastic means. The skincare agent is protected from bacteria by this storage and is also prevented from migrating during storage and transport.

Only when the absorbent article is put on, when the elastic means are stretched and their structure is thus altered and compressed, is the skincare agent pressed out through the structure of the elastic means. Transfer of the skincare agent then takes place in the areas where the elastic, and consequently the article, lie tightest against the user, i.e. where the risk of chafing and irritation is greatest. The majority of the skincare agent is expected to be pressed out to adjacent skin on the first stretching of the elastic means. However, a further advantage of the invention is that remaining skincare agent will be pressed out continuously during use as the elastic means will be further stretched when the user stretches and moves. A further advantage of the invention is that, as the skincare agent is safely stored in the elastic means, a greater range of skincare products can be used by using conventional elastic means which do not interact with the skincare product, whereupon a minimal effect on the function of the skincare agent is achieved.

Functional elastic means is here intended to mean such elastic means as are conventionally used in absorbent articles with the ability to hold the article in close proximity against the body of the user. In this connection, elastic means referred to are able to independently maintain necessary elastic function and which thus have a high elasticity module. At the same time, the invention is aimed at obtaining a cost-effective solution, which is important in disposable products, such as absorbent articles. For example, various elastic hydrogels, which do not have an elasticity module as required for the purpose and which, in addition, as a rule constitute a considerable cost increase for disposable articles, are not included. The elastic means used in the invention thus offer cost effectiveness as a result of the simpler handling in the process, i.e. the addition of skincare agent to an absorbent article in an easily manageable and safe manner. This is due to the fact that conventional elastic means are used in the process, for which means there already exists adapted equipment, and that release of the skincare agent is not obtained until during use.

Examples of conventional elastic means are those that are attached as leg elastic and waist elastic and which usually exist in the form of threads, bands, films, laminate or the like. Elastic means in absorbent articles described in the patent literature also exist in the form of side panels, raised elastic barriers known as standing gathers, belts, elastic edge elements, wings and front and rear body panels. When elastic threads or bands are used, two or more are often arranged parallel to one another and they then constitute part members in the elastic means. Similar elastic threads or bands can also be arranged as side barrier elastic in side barriers, which are usually placed on the inside of the leg elastic. The side barriers normally extend in a longitudinal direction along the absorption body and have a longitudinal edge which is attached to the absorbent article and a second free edge intended to lie against the user. In addition, elastic threads and bands can be arranged in elastic systems around, preferably, the crotch area.

The elastic component can be made of natural rubber, polybutadiene, low-crystalline polythene, metallocene-catalysed low-crystalline polythene, ethylene vinyl acetate copolymers (EVA), polyurethane, polyisopropene, butadiene styrene, copolymers and/or styrene block copolymers, especially styrene/isoprene/styrene (SIS), styrene/butadiene/styrene (SBS) or styrene/ethylenebutadiene/styrene-block copolymers and/or compounds of the said polymers. These materials or material compounds have high extensibility and are therefore suitable for the purpose. Further suitable elastic materials are described in WO 2004/021949, WO 03/047488, EP 0 521 883, EP 0 582 596, WO 97/20091 and U.S. Pat. No. 5,539,056.

According to one preferred embodiment of an absorbent article in accordance with the invention, the elastic means is essentially non-water absorbent, i.e. not absorbent of liquid such as water or urine. The elastic means advantageously absorbs less than 15% water or urine, preferably less than 10% water or urine. On the other hand, the elastic means can swell in a cream or lotion such as oil and in this way take up skincare agent in its network structure.

Essentially non-waterabsorbent thus denotes a material which absorbs less than 20% water or urine, preferably less than 15% water or urine and most preferably less than 10% water or urine. Essentially non-waterabsorbent is also intended to mean that the elastic means shall not take up or retain urine in such an amount that it adversely affects the function of the elastic means as a depot for skincare agent after wetting has taken place. The elastic means shall thus suitably be of non-waterabsorbing character. However, on a purely practical level, it is in principle unavoidable that one or a few drops of liquid would not be retained in the elastic network structure due to the capillary forces which occur in various cavities in the structure. However, this is not desirable and is minimised in accordance with the said structure being essentially non-waterabsorbent.

Accordingly, the elastic means is advantageously essentially non-waterabsorbent. This is so that absorption of moisture or fluid shall not press out skincare agent, for example before use so that the pores or holes in adjacent water-absorbing layers are blocked and thus prevent the fluid from penetrating into the absorption body. The liquid should also be able to be transported past the elastic means during repeated wetting without being absorbed and retained in the elastic means. Thus, liquid, for example, sweat, urine or blood should be able to pass into the absorbent body of the absorbent article in a free and relatively unrestrained manner. Due to the elastic means being non-waterabsorbent, liquid such as excreted urine or sweat are prevented from being absorbed in the structure and causing the elastic means to swell. Swelling would lead to already vulnerable areas being subjected to even more serious chafing and moisture. Furthermore, absorption of water, as mentioned above, can trigger release of skincare agent in less advantageous situations, for example during storage in a damp environment. A substantial release of urine could also cause the skincare agent to be pressed out in such a way that it is removed from the area where it is intended to act, i.e. nearest adjacent skin. Another advantage of using essentially non-waterabsorbent elastic means is that a greater amount of skincare agent can be used as the release is not dependent on factors such as temperature or moisture sensitivity.

According to another preferred embodiment of an absorbent article in accordance with the invention, the elastic means is constituted by a material with a recovery degree after loading of ca. 70-100%, preferably 80-100% and most preferably 90-100% directly after loading has ceased.

The degree of recovery can be measured using a simple method where: the length (L₀) of the elastic means is measured, whereafter the elastic means is subjected to a load of 200 kPa (≈49.8 g/cm²) during a predetermined period of time. The length L₁ is then measured directly after loading has ceased. Directly is intended here to mean ca. one minute.

In one embodiment of the absorbent article according to the invention the elastic means is chemically cross-linked rubber, such as natural rubber or styrene butadiene rubber or physically cross-linked rubber known as thermo(plastic) elastics, for example polyurethane rubber or thermoplastic olefines.

These constitute examples of elastic means conventionally used in absorbent articles, which means have high elasticity and good recovery capacity.

Natural rubber and styrene butadiene rubber constitute examples of morphologically homogeneous material in which covalent bonds bind together the polymer chains and leave free volume in the network. The skincare agent is incorporated in these materials by swelling the polymer chains and/or filling up the free volume. Release of the skincare agent is achieved in connection with stretching when the mechanical forces compress the free volume. A thermoplastic elastic such as polyurethane constitutes an example of a heterogeneous morphological structure with so-called hard and soft phases. The so-called hard phases are built up of chemical structures which can give the material flexibility. In the so-called soft phases the skincare agent can be inserted by swelling the polymer chain and/or filling up the free volume. The skincare agent is released when the soft phases are stretched out during use of the absorbent article.

According to one embodiment of the absorbent article, the elastic means comprises up to 4 g, preferably up to 1 g, more preferably up to 0.5 g and most preferably up to 0.1 g skincare agent/g elastic means in non-stretched condition. A greater interval limit is particularly preferred when elastic means in the form of superabsorbent oil-absorbents made of polyolefines are used. Materials such as polypropylene or polyethylene are particularly useful when incorporating up to about 1 g skincare agent/g. elastic means. However, it is advantageous to use not more than 0.5 g and most preferable to use not more than 0 μg of skincare agent/g. of elastic means in order to avoid the elastic means feeling sticky. The invention is thus also characterised in that it is possible to take up a limited amount of skincare agent in the elastic means, but nevertheless an amount that is suitable for the purpose. A controlled amount of skincare agent ensures that no excessive amounts of skincare agent are emitted during the first stretching and at the same time costs for use of a surplus of often expensive skincare agent are kept down. A surplus of skincare agent also involves an increased risk of migration of the said means to the absorption core of the absorbent article.

The skincare agent according to the invention is preferably an oil, lotion, ointment, anti-chafing means, film-building polymers, wax or substances deriving from the plant kingdom or the like. Furthermore, the skincare agent is preferably suitable for use as a preventive, soothing or healing means for dermatitis. In a further embodiment, the skincare agent is suitable for preventing or reducing chafing and/or irritation. In a further embodiment of the invention, the skincare agent comprises pH-regulating substances, anti-microbial substances, glucocorticoids, anti-viral means, probiotic microorganisms, enzyme-inhibiting agents, substances which are active to varying degrees, e.g. anti-inflammatory substances. As has been mentioned above, it is the case that when the user has sensitive skin and may get or already has a rash, irritation or bedsores, it is important that the surface of the sensitive skin is not subjected to chafing. Besides the fact that chafing purely physically can inflict surface damage to the skin, chafing also makes the skin more susceptible to other superficial influences such as faeces, urine and the extreme conditions which occur as a result. This problem is solved by the skincare agent comprising one of the above components.

### BRIEF DESCRIPTION OF DRAWINGS

The invention will be described below in greater detail with reference to the figures shown on the attached drawings, in which

FIG. 1 shows a frontal view of a pant diaper as it appears when put together,

FIG. 2 shows a diaper according to a further embodiment of the invention and

FIG. 3 shows a cross-section along the line II-II through the diaper in FIG. 2.

### DESCRIPTION OF EMBODIMENTS

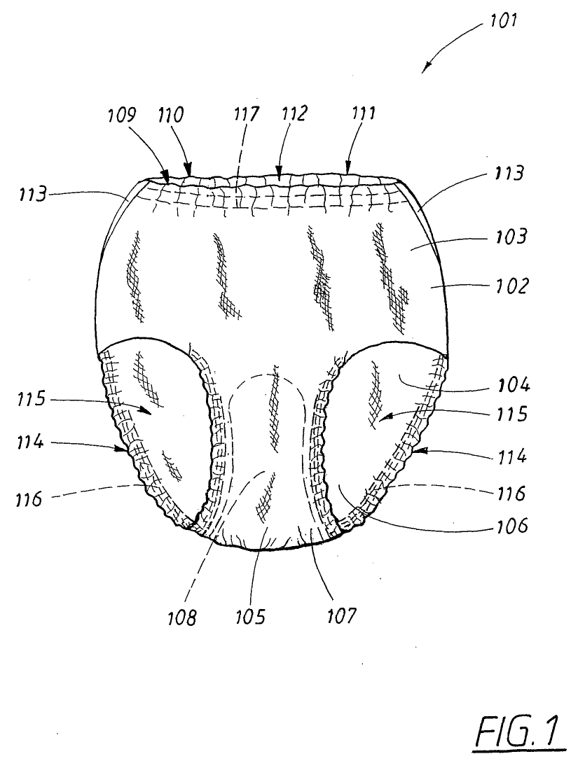

The pant diaper 101 in FIG. 1 is shown in assembled form and without being subjected to stretching forces. The pant diaper comprises an outer pant 102, which can be divided into a front portion 103, which during use is intended to be directed forwards on the user and to be arranged over the user's stomach, a rear portion 104, which during use is intended to be directed rearwards on the user and to be arranged over the user's buttocks, and a crotch portion 105 situated between the front portion 103 and the rear portion 104, which is intended to be arranged in the user's crotch during use.

The outer pant 102 shown in FIG. 1 comprises a first liquid-permeable cover layer 106, a second liquid-impermeable cover layer 107 and an absorption body 108 arranged between the cover layers 106, 107. The liquid-permeable cover layer 106 can be constituted of any material suitable for the purpose, such as layers of nonwoven material, perforated plastic film, net material, tow (parallel fibres), or the like. The cover layer 106 can of course also consist of a laminate of two or more layers of the same or different material. The liquid-impermeable cover layer 107 can consist of a liquid-impermeable plastic film, a nonwoven layer which has been coated with a liquid-barrier material, or some other easily flexible material layer which has the ability to resist liquid penetration. The liquid-impermeable cover layer 107 can also consist of a laminate. However, it can be an advantage if the liquid-impermeable cover layer 107 displays a certain breathability, i.e. allows the passage of water vapour through the layer 107.

The absorption body 108 can be constructed of one or several layers of absorbent material such as cellulose fluff pulp, tissue, absorbent foam, etc. It is also common for the absorption body to contain superabsorbents, i.e. polymer materials which can absorb body fluid equivalent to several times their own weight while forming a hydrogel. Such superabsorbents usually exist in the form of particles, but fibres, flakes, granulates, foam and film are also found. Furthermore, the absorption body 108 can also comprise non-absorbent components, such as stiffening elements, forming elements, binding agents, etc.

In assembled form, as is shown in FIG. 1, the respective side edges of the diaper are put together in order to form a side seal 113. The front edge 109 and rear edge 110 of the pant diaper 101 form a waist edge 111, which surrounds a waist opening 112 and the leg segments of the side edges form leg edges 114 which surround leg openings 115.

Elastic means are arranged as leg elastic 116 around the leg openings 115 and as waist elastic 117 around the waist openings 112. As the outer pant 102 is formed of a laminate of two or more layers, the elastic means 116, 117 can be attached between two such layers. Alternatively, the elastic means can be arranged so that they lie directly against the user's skin during use. The leg elastic 116 is arranged only on the crotch portion 105 and the rear portion 104, while the part of the leg edges 114 which extends over the front portion 103 is free from any particular leg elastic but can expediently extend the entire distance around the leg openings 115. Further, the outer pant 102 comprises waist elastic 117 around the waist opening 112 in order to ensure that the pant diaper 102 sits securely and comfortably in place and fits tightly around the user's waist.

The elastic means in the leg elastic 116 and waist elastic 117 is in the form of elastic threads, bands, or the like. When elastic threads or bands are used, two or more of them are often arranged parallel to each other and constitute elastic part members in the elastic means.

As can be seen in FIG. 1, the number of elastic part members in the leg elastic 116 is greater along the side edges of the absorption packet in the crotch area than along other parts of the leg edges 114. This is a suitable embodiment in order to facilitate the forming and curving of the absorption body 108, as the curving resistance in the absorption body 108 is greater than in the more flexible surrounding parts of the pant diaper. This is also particularly advantageous in view of the fact that the risk of chafing is particularly evident in this area and thus several elastic means comprising the skincare agent can advantageously be placed in this area.

As has been mentioned previously, FIG. 1 shows how the pant diaper appears before it is put on to the user. All elastic means and components are essentially unstretched and the skincare agent is then safely stored until the pant diaper is stretched during use. This means that before it is put on, the pant diaper 101 displays a somewhat puckered waist edge 111 and also leg edges 114 which are puckered at the crotch portion 105 and the rear portion 104.

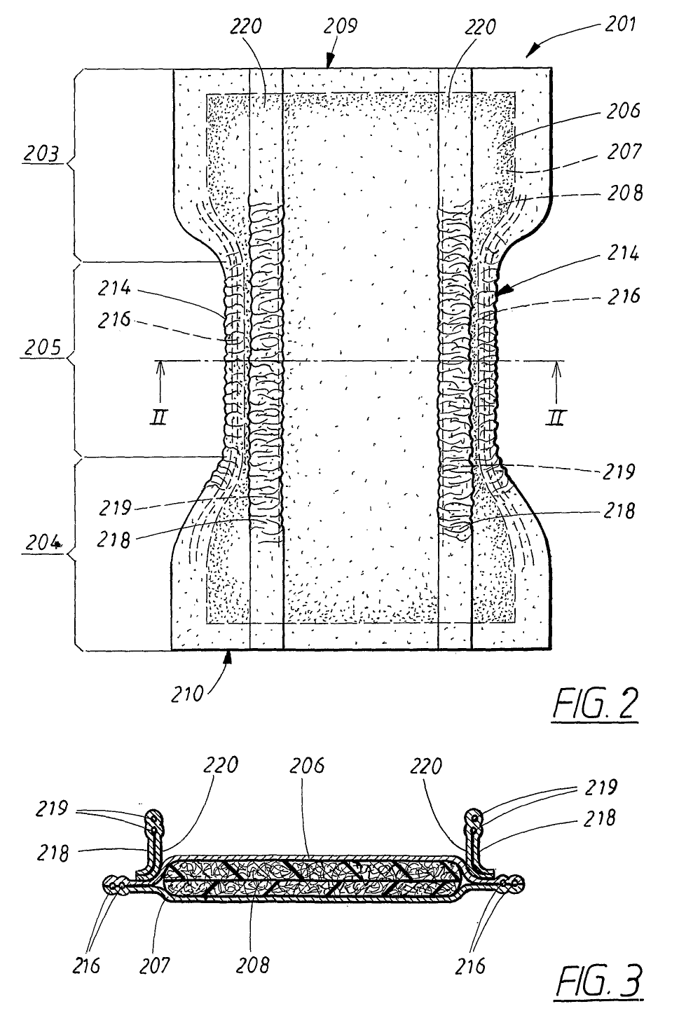

FIG. 2 shows a diaper which, for the sake of clarity, is in a “stretched out”, relatively plane condition. The elastic means are thus also shown in stretched out condition here. FIG. 3 shows a cross-section of the diaper in the line II-II. The diaper 201 shown in FIG. 2 shows, in a corresponding manner to the pant diaper shown in FIG. 1, a front portion 203, a rear portion 204 and a narrower, intermediate crotch portion 205. Furthermore, the diaper 1 has two longitudinal, inwardly curved side edges 214, a front edge 209 and a rear edge 210. The diaper in FIGS. 2 and 3 also comprises a first, liquid-permeable cover layer 206, a second liquid-impermeable cover layer 207 and an absorption body 208 arranged between the cover layers 206, 207. The two cover layers 206, 207 have a greater extension in the plane than the absorption body 208 and project out beyond the absorption body 108 around the whole of its periphery. The cover layers 206, 207 are mutually connected within the projecting parts, for example by means of gluing or welding with heat or ultrasound.

Further, the diaper 201 is provided with longitudinal elastic means 216, arranged prestretched along the side edges 214 of the diaper. These elastic means 216 contribute to curving the diaper 201 in accordance with the user's body during use and at the same time constitute the leg elastic of the diaper. In this way, the leg elastic 216 serves to hold the side edges 214 of the diaper in sealing contact against the user's legs in order to prevent the occurrence of gaps between the diaper and the user's body during use, through which gaps body fluid can leak out of the diaper.

The diaper 201 also has inner elastic barriers 218. The inner elastic barriers 218 consist of doubled material strips 220. The material strips 220 suitably consist of a material which is able to resist liquid penetration, for example, hydrophobic nonwoven, plastic film or laminate of nonwoven and plastic film, at the same time penetrable for the hydrophobic skincare agent. Each material strip 220 is attached to the liquid-permeable cover layer 206 along an attachment edge and comprises side barrier elastic 219 in the form of elastic part members, in this case elastic threads, at an opposite fold edge.

However, it is in accordance with the invention that the folded material strips 220 are essentially not urine-absorbent and therefore the material on at least the surface which lies closest to the user's skin should be constituted by an essentially hydrophobic material, for example synthetic fibres such as polyolefin fibres which have not been treated with wetting means. Those parts of the cover layers which surround the leg elastic 216 can be treated in a corresponding manner. Naturally, both the leg elastic 216 and the side barrier elastic 219 can be attached outside the folded material strips 220 or over the liquid-permeable cover layer 206, respectively, in order to thus come into closest contact with the user's skin during use.

What is described above should not be considered to limit the invention to use together with only those absorbent articles which are described in these examples. All forms of absorbent article in which elastic means are used which are known to a person skilled in the art of diapers, incontinence products, sanitary napkins, panty shields, or the like should be considered to be included. Furthermore, the invention shall not be limited to the elastic means exemplified above, but can also include elastic belts attached to absorbent articles, wings in sanitary napkins, etc.

### Diaper Dermatitis

Several factors in combination lead to the development of diaper dermatitis. Moist skin results in skin being more easily worn down by chafing and pressure. A high moisture content also means that irritants can penetrate the skin to a greater extent and also that bacteria and fungi thrive better. Occlusion of skin and breaking down of the urea in urine to ammoniac results in an increase in pH. The increased pH-value leads to enzymes (lipases and proteases), which come from the bowel and from microorganisms in the faeces, being able to break down the skin to a greater degree. A vicious circle can easily develop in which various factors aid and intensify each other.

The best way to prevent dermatitis is to create conditions which counteract the factors which create and maintain the diaper dermatitis process. The aim should therefore be to keep the skin as dry as possible, to air the skin often and to change wet diapers. Mechanical shearing forces should be minimised by choosing materials that are as smooth and soft as possible and by reducing abrasion between diaper and skin. Applying a softening and protective lotion or cream to the skin makes it possible to reinforce the barrier against penetration of irritants and enzymes. In more serious cases of dermatitis, microorganisms can have infected the damaged skin and treatment with more active medicines is necessary. In those cases, ointments with cortisone and various anti-fungal and anti-bacterial means are used.

### Examples of Skincare Agents for Use in Elastic Means

Skincare agent can be used to prevent, relieve or heal dermatitis. A skincare agent can, in its physical form, be constituted by a solution, suspension, cream, lotion, ointment, paste, gel, foam, aerosol, etc. In the following description cream, lotion or ointment are preferably used, but other forms described above are, of course, also conceivable.

Skincare agents can include lipids (fats, oils, waxes), solvents (including water), water-soluble substances, surface-active agents (emulsifiers, surfactants), viscosity-regulating substances, pH-regulating substances, preserving agents, complexing agents (e.g. chelates), delivery systems (e.g. liposomes, microcapsules, etc), pigments, perfumes, and active substances (also pharmaceutical agents). The lipids are usually emulsified in water, known as o/w emulsion, or water is emulsified in the lipid phase, known as w/o emulsion.

Skincare agents can include lipids such as:

paraffins (alkanes) with 12-35 carbon, for example paraffin oil (mineral oil) or petrolatum (vaseline).

Triglycerides, refined and/or hydrogenated, animal or vegetable with preferably carbon chain lengths of under C-18 (e.g. milk fat, coconut oil Cocous nocifera, palm-kernel oil Elaeis guineeis), animal or vegetable with unsaturated C-18 fatty acids (e.g. Japan wax Rhus succesdanes, tallow fat, soybean oil Glycerin soya, peanut oil Arachais hypogaea, maize oil Zea mays, sunflower oil Helanthus annus, grapeseed oil Vitis vinifera, safflower oil Carthamus tinctorius, sweet almond oil Prunnus amygdalus dulcis, hazelnut oil Corylus americana, walnut oil Juglans regia, olive oil Olea europasa, avocado oil Persea gratissima, sesame oil Sesamum indicum, tall oil, Tallol, cottonseed oil Gopssypium, palm oil Elaesis guineensis, rice oil Oryza sativa, rape oil Canola, apricot-kernel oil Prunus armeniaca, cocoa butter Theobroma cao, shea butter Butyrospermum parkii, wheatseed oil Triticum vulgare, Bassia latifola), animal or vegetable with carbon chains over C-18 (e.g. beeswax Cera alba, shellac wax Shellac cera, meadowfoam seed oil Limnanthes alba, rapeseed oil Brassica capmestris, cucumberseed oil Borago officinalis, linseed oil Linum usitatissimum, ricin oil Ricinus communis, veronia oil Veronia galamensis, jojoba oil Buxus chinensis, candlewax Euphorbia cera, ongokea oil Ongokea gore).

Fatty alcohols with straight or branched carbon chain lengths of 12-32 carbons. For example, cetyl alcohol or stearyl alcohol.

Fatty acid esters with 12-32 carbons. For example, methyl palmitate, methyl stearate, isopropyl myristate, isopropyl laurate, isopropyl palmitate, isopropyl stearate, octyl palmitate, octyl stearate or octyl laurate.

Polyalcohols. For example, sugar alcohols or polyglycerols.

Complex lipids. For example, phospholipids or sphingolipids (ceramides).

Waxes. Of animal origin, for example beeswax or lanolin. Of vegetable origin, for example carnauba or candelilla. Of mineral origin, for example ozocerite or ceresin.

Polysiloxanes. Straight, branched or cyclic. For example, polydimethylsiloxane (dimethicone) or polydiethylsiloxane.

Skincare agents can include emulsions such as:

Emulsions of one or more fats with hydrophilic substances such as water, glycerol, polyethylene glycol (PEG), propylene glycol, butylene glycol, sorbitol, silicone glycols or the like or mixtures thereof.

Skincare agents can include substances which adsorb irritating components in urine or excrement. For example, clay mineral (bentonite, kaolin, montmorillonite, etc), silicon oxide compounds (quartz, zeolites, water glass, etc) or activated charcoal. The substances can advantageously have been activated to be more adsorbent by means of various treatments, for example with quaternary ammonium compounds.

Skincare agents can include enzyme inhibitors. For example, metal salts of iron or zinc, trace amounts of heavy metal ions such as copper or silver, ethylene diamine tetraacetic acid (EDTA), soybean trypsin inhibitor, lima bean protease inhibitor, maize protease inhibitor, stearylglycyrrhetinate, glycerol triacetate, betaine compounds, sulphobetaine compounds, cholestyramine, p-guanidinobenzoates.

Skincare agents can include pH-regulating additives. For example, organic or inorganic acids such as adipic acid, ascorbic acid, benzoic acid, citric acid, malic acid, tartaric acid, lactic acid, phosphoric acid or hydrochloric acid. Or buffers made for example from said acids with corresponding salts. Can also include polymeric acids, for example polyphosphoric acid or polyacrylic acid.

Skincare agents can also include additions of probiotic microorganisms, characterized by being antagonistic towards undesired microorganisms, e.g. urinary tract pathogens or skin infection pathogens. Examples of probiotic microorganisms which can be used are individual strains or mixtures of several strains of lactic acid bacteria taken from the species Lactobacillus acidophilus, Lactobacillus curvatus, Lactobacillus plantarum or Lactococis lactis. Skincare agents can also comprise prebiotic substances such as glycogene.

Skincare agents can also include more or less active substances such as:

Anti-inflammatory agents, e.g. acetylsalicylic acid, allantoin, azulen, alpha-bisabolol (chamomile), flavonoids, glycyrrhizinic acid, ichthammol (Inotyol®), tannins.

Astringents (vasoconstrictors), for example TiO, ZnO (and other Zn compounds), aluminium acetate solution, aluminium tartrate solution (and other Al compounds), ethanol or ethanol-based solutions.

Aloe vera (Aloe barbadensis), alpha-hydroxy acids (citric acid, tartaric acid, lactic acid, malic acid, etc.), algae extract, ascorbic acid (vitamin C), vitamin A compounds (retinol, retinal, tretinoin and isotretinoin), avocado sterols, betaine (trimethylglycine), ceramides, grapeseed extract, essential fatty acids, flavonoids, phytosphingosine, phytosterols, hyaluronic acid, yeast extract, chitosan, milk protein (Lactis proteinum), pantenol (provitamin B5), polysaccharides, rosemary extract, tocopherol (vitamin E), ubiquinone (coenzyme Q10), urea.

Antimicrobial agents, for example amorolfin, antibiotics, bacitracin, benzalkonium chloride, benzetonium chloride, cetrimide, fusidic acid, gentian violet (methylrosaniline chloride), hexachlorophene, hexylresorcinol, imidazole derivatives (for example biphonazole, econazole, ketoconazole, chlotrimazole, miconazole), chlorhexidine, nystatin, povidone-iodine, terbinafin, triclosan, hydrogen peroxide.

Metals and their salts, e.g. Ag, Cu, Zn, Mg, Na.

Antiviral agents, for example acyclovir, imiquimod, podophyllotoxin, podophilox, cidofovir, penciclovir, vidarabin, idoxuridine, trifluridine, tromantadine, lamivudine.

Skincare agents can also include glucocorticoids, preferably of low potency, for example hydrocortisone, or antipruritic, for example antihistamines or local anaesthetics (e.g. lidocaine).

Skincare agents can also consist of ready-made mixtures of skin ointments, creams and lotions. For example, Necesse® Lotion (ingredients: aqua, propylene glycol, liquid paraffin, octyl octanoate, urea, PEG-8 distearate, steareth-2, steareth-21, betaine, lactic acid, tocopheryl acetate, dimethicone, tromethamine, methylparaben, propylparaben, perfume), Necesse® Skin Cream (ingredients: aqua, liquid paraffin, octyl stearate, sodium chloride, urea, glyceryl stearate, stearic acid, cetearyl alcohol, PEG-30 stearate, tocopheryl acetate, tromethamine, dimethicone, methylparaben, sorbic acid, propylparaben, perfume), Necesse® Barrier Cream (ingredients: petrolatum, glycerol, Arachis hypogaea, triethyl citrate, tocopheryl acetate) or Necesse® Zinc Ointment (ingredients: petrolatum, Arachis hypogaea, zinc oxide, retinyl palmitate, tocopherol). Necesse® products are sold commercially by SCA Hygiene Products, Gothenburg, Sweden.

Other examples of some different skincare agents and/or substances that can be used with the invention are described partially inter alia in the following documents: WO 96/16682 “Diaper having a lotioned topsheet” (Roe et al.), WO 96/16681 “Diaper having a lotioned topsheet containing a polysiloxane emmollient” (Roe, Mackey), WO 97/05909 “Diaper having a lotioned topsheet comprising a liquid polyester emollient and an immobilizing agent” (Roe), WO 99/45973 “Disposable absorbent article having a skin care composition containing an enzyme inhibitor” (Roe et al.), WO 99/45974 “Protease inhibitors in absorbent products” (Rourke et al.), WO 99/45976 “Proton donating actives in absorbent articles” (McOsker et al.), DE 33 09 530 C1 “Hygienische Absorptionsvorlage” (Leitner et al.), DE 41 36 540 A1 “Einwegwindeln” (Grunecker et al.), U.S. Pat. No. 3,489,148 “Topsheet for disposable diapers” (Duncan et al.), WO 00/64502 “Absorbent article having a lotionized bodyside liner” (Krzysik et al.), WO 00/64501 “Skin-friendly absorbent articles and compositions” (Krzysik et al.), WO 00/64500 “Absorbent article having a hydrophilic lotionized bodyside liner” (Krzysik et al.), WO 00/64503 “Skin-friendly absorbent articles and compositions” (Krzysik et al.), WO 99/22684 “Web materials with two or more skin care compositions disposed thereon and articles made therefrom” (Roe et al.).

It should be noted that the invention is not limited to the skincare agents mentioned above, these merely being examples of what could be used and that the invention

1. Absorbent article, comprising one or several parts each of which include elastic means, wherein the elastic means holds a skincare agent, wherein the skincare agent is arranged to be released when the elastic means is stretched during use of the absorbent article, and wherein the elastic means absorbs less than 20% water or urine

2. Absorbent article in accordance with claim 1, wherein the elastic means absorbs less than 15% water or urine.

3. Absorbent article in accordance with claim 1, wherein the elastic means is constituted by a material with a recovery degree after loading of ca. 70-100% directly after loading has ceased.

4. Absorbent article in accordance with claim 1, wherein the elastic means is incorporated into the absorbent article as a waist elastic, a leg elastic, a side barrier elastic, a side panel, a raised elastic barrier, a belt, an elastic side element, a wing, a front body panel or a rear body panel.

5. Absorbent article in accordance with claim 1, wherein the elastic means is natural rubber or a thermoplastic elastic.

6. Absorbent article in accordance with claim 1, wherein the elastic means comprises up to 4 g skincare agent/g elastic means in non-stretched condition.

7. Absorbent article in accordance with claim 1, wherein the skincare agent is an oil, lotion, ointment, anti-chafing means, film-building polymers, wax or substances deriving from the plant kingdom.

8. Absorbent article in accordance with claim 1, wherein the skincare agent is suitable for use as a preventive, soothing or healing means for dermatitis.

9. Absorbent article in accordance with claim 1, wherein the skincare agent is suitable for preventing or reducing at least one of chafing and irritation.

10. Absorbent article in accordance with claim 1, wherein the skincare agent comprises pH-regulating substances, anti-microbial substances, glucocorticoids, anti-viral means, probiotic microorganisms, or enzyme-inhibiting agents.

11. Absorbent article in accordance with claim 1, wherein the skincare agent comprises an anti-inflammatory substance or a substance derived from the plant kingdom.

12. Absorbent article in accordance with claim 1, wherein the absorbent article is a diaper, an incontinence product, a sanitary napkin, or a panty liner.

13. Absorbent article in accordance with claim 2, wherein the elastic means absorbs less than 10% water or urine.

14. Absorbent article in accordance with claim 3, wherein the recovery degree after loading is 80-100% directly after loading has ceased.

15. Absorbent article in accordance with claim 14, wherein the recovery degree after loading is 90-100% directly after loading has ceased.

16. Absorbent article in accordance with claim 5, wherein the elastic means is polyurethane, polybutadiene, low-crystalline polythene, metallocene-catalysed low-crystalline polythene, ethylene vinyl acetate copolymers (EVA), polyurethane, polyisopropene, butadiene styrene, at least one of copolymers and styrene block copolymers, or compounds of the said polymers.

17. Absorbent article in accordance with claim 16, wherein the at least one of copolymers and styrene block copolymers is styrene/isoprene/styrene (SIS), styrene/butadiene/styrene (SBS) or styrene/ethylenebutadiene/styrene-block copolymers.

18. Absorbent article in accordance with claim 6, wherein the elastic means comprises up to 1 g skincare agent/g elastic means in non-stretched condition.

19. Absorbent article in accordance with claim 18, wherein the elastic means comprises up to 0.5 g skincare agent/g elastic means in non-stretched condition.

20. Absorbent article in accordance with claim 18, wherein the elastic means comprises up to 0.1 g skincare agent/g elastic means in non-stretched condition.
